SEQUENCE LISTING (6 sequences)

SEQ ID NO: 1 - DNA, 746 bp, Homo sapiens
  CDS (56)...(442)
ccagcaggaggcacaggaaaactgcaagccgctctgttcctgggcctcggaagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactagcccatcctctcccctccttcctcccctgggtcacagggcttgacattctggtgggggaaacctgtgttcaagattcaaaaactggaaggagctccagccctgatggttacttgctatggaatttttttaaataaggggagggttgttccagctttgatcctttgtaagattttgtgactgtcacctgagaagaggggagtttctgcttcttccctgcctctgcctggcccttctaaaccaatctttcatcattttacttccctctttgcccttacccctaaataaagcaagcagttcttga

SEQ ID NO: 2 - protein, 129 aa, Homo sapiens
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 3 - protein, 106 aa, Homo sapiens
QEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 4 - DNA, 390 bp, Homo sapiens
  CDS (1)...(390)
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatc

SEQ ID NO: 5 - protein, 130 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 6 - protein, 106 aa, Homo sapiens
ASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

(19) United States
(12) Patent Application Publication
(10) Pub. No.: US 2005/0272635 A1
(43) Pub. Date: Dec. 8, 2005

(54) Use of corticotroph-derivced glycoprotein hormone to treat liver steatosis

(76) Inventor: James D. Kelly, Mercer Island, WA (US)

Correspondence Address: Robyn Adams;ZymoGenetics, Inc., 1201 Eastlake Avenue East, Seattle, WA, 98102, US

(21) Appl. No.: 11/073,222

(22) Filed: Mar. 4, 2005

Related U.S. Application Data
(60) Provisional application No. 60/550,623, filed on Mar. 5, 2004.

(51) Int. Cl. A61K 38/17

(52) U.S. Cl. 514/2

(57) ABSTRACT

The use of corticotrph-derived glycoprotein hormone to treatment liver steatosis, liver steatohepatitis, and cirrhosis of the liver, as well as other abnormalities in hepatic function that are related to obesity.

### CROSS REFERENCE TO RELATED APPLICATIONS

This application claims the benefit of U.S. Provisional Application Ser. No. 60/550,623, filed Mar. 5, 2004, which is herein incorporated by reference.

### FIELD OF THE INVENTION

The present invention relates to the treatment of liver steatosis, liver steatohepatitis, and cirrhosis of the liver, as well as other abnormalities in hepatic function that are related to obesity, using corticotroph-derived glycoprotein hormone.

### BACKGROUND OF THE INVENTION

Although liver disease is not a widely appreciated complication of obesity, epidemiologic evidence suggests that obesity increases the risk of cirrhosis. For example, in autopsy series, obesity was identified as the only risk factor for disease in 12% of cirrhotic subjects. See Yang, S. Q. et al. (1997) Proc Natl Acad Sci USA 94, 2557-2562. Notably, cirrhosis is approximately six times more prevalent in obese individuals than in the general population. In the USA, the high percentage of overweight people in the general population partially explains the fact that non-alcoholic fatty liver disease (NAFLD) is the most common liver disease. Type-2 diabetes is present in 33% of these subjects. The degree of obesity correlates positively with the prevalence and severity of fatty liver (steatosis), and this in turn correlates with steatohepatitis. A current explanation of the pathogenesis of steatohepatitis is the “two-hits” hypothesis. See Day, C. P, and James, O., Gastroenterology 114, 842-845. The first “hit” is the depositing of fat in hepatocytes, leading to fatty degeneration of the liver or steatosis hepatitis. This fatty degeneration increases the organ's sensitivity to the second “hit”, which can be any one of a variety of insults including diabetes, lipid peroxidation due to drug metabolism or excess alcohol intake.

Thus, there is a need for treating liver steatosis, steatohepatitis, cirrhosis of the liver, and liver abnormalities associated with obesity. The present invention fills this need by administration of a novel glycoprotein hormone to individuals with these diseases.

### DESCRIPTION OF THE INVENTION

In an aspect, the invention provides a method for treating steatosis in a mammal, comprising administering to the mammal a pharmaceutically effective amount of a CGH polypeptide, wherein administration of the polypeptide results in a reduction in the steatosis. In an embodiment, said mammal is obese. In another embodiment, said mammal is type-2 diabetic. In another embodiment, said mammal has metabolic syndrome. In another embodiment, said mammal is hyperlipidemic. In another embodiment, said mammal is insulin resistant.

In another aspect, the invention provides a method for treating steatohepatitis in a mammal, comprising administering to the mammal a pharmaceutically effective amount of a CGH polypeptide, wherein administration of the polypeptide results in an improved steatohepatitic state. In an embodiment, said mammal is obese. In another embodiment, said mammal is type-2 diabetic. In another embodiment, said mammal has metabolic syndrome. In another embodiment, said mammal is hyperlipidemic. In another embodiment, said mammal is insulin resistant.

In another aspect, the invention provides, a method for preventing steatohepatitis in a mammal, comprising administering to the mammal a pharmaceutically effective amount of a CGH polypeptide, wherein administration of the polypeptide maintains or reduces the steatohepatitis. In an embodiment, said mammal is obese. In another embodiment, said mammal is type-2 diabetic. In another embodiment, said mammal has metabolic syndrome. In another embodiment, said mammal is hyperlipidemic. In another embodiment, said mammal is insulin resistant.

In another aspect, the invention provides, a method for preventing cirrhosis of the liver in a mammal, comprising administering to the mammal a pharmaceutically effective amount of a CGH polypeptide, wherein administration of the polypeptide maintains or reduces the cirrhosis. In an embodiment, said mammal is obese. In another embodiment, said mammal is type-2 diabetic. In another embodiment, said mammal has metabolic syndrome. In another embodiment, said mammal is hyperlipidemic. In another embodiment, said mammal is insulin resistant.

In another aspect, the invention provides a method for treating cirrhosis of the liver in a mammal, comprising administering to the mammal a pharmaceutically effective amount of a CGH polypeptide, wherein administration of the polypeptide reduces the cirrhosis. In an embodiment, said mammal is obese. In another embodiment, said mammal is type-2 diabetic. In another embodiment, said mammal has metabolic syndrome. In another embodiment, said mammal is hyperlipidemic. In another embodiment, said mammal is insulin resistant.

These and other aspects of the invention will become evident upon reference to the following detailed description of the invention.

### DETAILED DESCRIPTION OF THE INVENTION

The present invention comprises administering corticotroph-derived glycoprotein hormone (CGH) to an individual to reduce, prevent, or reverse liver steatosis, liver steatohepatitis, cirrhosis of the liver, as well as other hepatic abnormalities associated with obesity and type-2 diabetes. In one aspect, the individual is a mammal. In an embodiment, the mammal is human.

Corticotroph-derived glycoprotein hormone (CGH) is a heterodimeric protein hormone released from corticotroph cells in the anterior pituitary. CGH is disclosed in International Patent Application No. PCT/US01/09999, publication no. WO 01/73034. It is comprised of an alpha subunit, glycoprotein hormone alpha2 (GPHA2), and a beta subunit, glycoprotein hormone beta 5 (GPHB5). GPHA2 was previously called Zsig51 (International Patent Application No. PCT/US99/03104, publication no. WO 99/41377 published Aug. 19, 1999; U.S. Pat. No. 6,573,363). SEQ ID NO: 1 is the human cDNA sequence that encodes the full-length polypeptide GPHA2, and SEQ ID NO:2 is the full-length polypeptide sequence of human GPHA2. SEQ ID NO:3 is the mature GPHA2 polypeptide sequence without the signal sequence. SEQ ID NO:4 is the human cDNA sequence that encodes the full-length GPHB5 polypeptide. SEQ ID NO: 5 is the full-length GPHB5 polypeptide. SEQ ID NO: 6 is the mature GPHB5 polypeptide without the signal sequence. The present invention also includes CGH polypeptides, and polynucleotides, that are substantially homologous to those of the SEQ ID NOs: 1,2,3,4,5, and 6.

The teachings of all of the references cited herein are incorporated in their entirety herein by reference.

GPHA2 is 25% identical in amino acid sequence to the common alpha subunit of the known glycoprotein hormones, and is predicted to have similar structural motifs. GPHB5 is approximately 30% identical in sequence to the beta subunits of human chorionic gonadotropin, thyroid-stimulating hormone, follicle-stimulating hormone, and luteinizing hormone, and is also predicted to be structurally conserved.

GPHA2 does not dimerize with any of the other glycoprotein hormone beta subunits, nor does GPHB5 dimerize with the common alpha subunit. As shown in Example 3, when co-expressed in the same cell, GPHA2 and GPHB5 form a non-covalent heterodimer, CGH, which contains two N-linked glycosylations on the GPHA2 subunit and one N-linked glycosylation on the GPHB5 subunit.

CGH exerts its effects through interaction with the thyroid-stimulating hormone (TSH), or thyrotropin, receptor. See Nakabayashi, K., et al. (2002) J Clin Invest 109, 1445-1452. The TSH receptor (TSHR) is a member of the G-protein coupled, seven-transmembrane receptor superfamily. Activation of the TSH receptor leads to coupling with heterotrimeric G proteins, which evoke downstream cellular effects. The TSH receptor has been shown to interact with G proteins of subtypes Gₛ, G_{q}, G₁₂, and Gᵢ. In particular, interaction with Gₛ leads to activation of adenyl cyclase and increased levels of cAMP. See Laugwitz et al., Proc Natl Acad Sci USA 93: 116-20 (1996). Elevation of cAMP levels leads to activation of protein kinase A, a multi-potent protein kinase and transcription factor eliciting diverse cellular effects. See Boume et al., Nature 349: 117-27 (1991).

The TSHR was originally identified in the thyroid as the principal activator of the thyroid gland, following exposure to the glycoprotein hormone, TSH. TSH release from the anterior pituitary stimulates the TSHR, resulting in secretion of thyroid hormone, stimulation of thyroid hormone synthesis, and cellular growth. TSH release is regulated by thyroid hormone levels, and is potently inhibited by elevated glucocorticoid levels. See, Utiger, in Endocrinology and Metabolism (Felig and Frohman, eds), pp. 261-347, McGraw-Hill, (2001).

Recently, the TSHR has been identified in many cell types not previously recognized, including cells of the immune system, brain, and reproductive organs. See, Example 1. Although the presence of TSH receptors in adipose tissue has been the subject of controversy for some time, recent reports have documented the presence of TSHR in adipose tissue of humans and rodents. Se, Bell, A., et al. (2000) Am J Physiol Cell Physiol 279, C335-340, and Endo, T., et al. (1995) J Biol Chem 270, 10833-10837.

CGH is a potent activator of the TSHR. In adipose cells, sub-nanomolar levels of CGH stimulate release of free fatty acids (FFA). Compared to TSH, CGH stimulates the release of FFA at 10-fold lower molar concentrations.

Treatment dosages should be titrated to optimize safety and efficacy.

Methods for administration include intravenous, peritoneal intramuscular, and topical.

Pharmaceutically acceptable carriers include but are not limited to, water, saline, and buffers. Dosage ranges would ordinarily be expected to be from 0.1 μg to 1 mg per kilogram of body weight per day. A useful dose to try initially would be 25 μg/kg per day, with the exact dose determined by the clinician according to accepted standards, taking into account the nature and severity of the condition to be treated, patient traits, etc. Within this dosage range, a dose of 5 μg/kg/day can be used. Also within this range, a range from 5 μg/kg/day to 100 μg/kg/day can also be used. However, the doses may be higher or lower as can be determined by a medical doctor with ordinary skill in the art. For a complete discussion of drug formulations and dosage ranges see Remington's Pharmaceutical Sciences, 17th Ed., (Mack Publishing Co., Easton, Pa., 1990), and Goodman and Gilman's: The Pharmacological Bases of Therapeutics, 9th Ed. (Pergamon Press 1996).

For pharmaceutical use, the proteins of the present invention can be administered orally, rectally, parenterally (particularly intravenous or subcutaneous), intracisternally, intravaginally, intraperitoneally, topically (as powders, ointments, drops or transdermal patch) bucally, or as a pulmonary or nasal inhalant. Intravenous administration will be by bolus injection or infusion over a typical period of one to several hours. In general, pharmaceutical formulations will include a CGH protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Doses of CGH polypeptide will generally be administered on a daily to weekly schedule. Determination of dose is within the level of ordinary skill in the art. The proteins may be administered for acute or chronic treatment, over several days to several months or years. In general, a therapeutically effective amount of CGH is an amount sufficient to produce a clinically significant change in an inflammatory condition.

NAFLD is now recognized to be an obesity-related metabolic syndrome. Although the pathogenesis of NAFLD has remained poorly understood, recent investigations have formed a clearer picture of the etiology of the disease. Insulin resistance is the most reproducible factor in the development of the disease. Insulin resistance is thought to lead to the accumulation of fat within hepatocytes, which then become substrates of mitochondrial reactive oxygen species for formation of reactive lipid peroxides, leading to fibrosis and steatohepatitis. Hyperlipidemia is a second risk factor for steatosis and NAFLD. About half of patients with hyperlipidemia were found to have NAFLD in one study. See Angulo, Paul, Nonalcoholic Fatty Liver Disease, New England Journal Medicine, Vol 346, p. 1221-1231.

CGH has been demonstrated to have activities that combat metabolic syndrome, including insulin sensitizing actions and anti-hyperlipidemic actions in models of obesity (See U.S. patent Publication No. 2003-0095983, published May 22, 2003, and see Example 3). Treatment of obese steatotic ob/ob mice with CGH resulted in the reversal of the steatotic state, whereas treatment with thyroid hormone resulted in no significant change in steatosis. High power images of liver sections of CGH-treated animals revealed areas comprising normal hepatocyte architecture, without significant accumulation of lipid. The action of CGH as an anti-steatotic, thus may be in part due to an overall beneficial effect on glucose, insulin and lipid levels, which, when disregulated, are the hallmarks of the metabolic syndrome.

Steatosis of any etiology can be associated with the development of fibrosis, so called steatohepatitis, and even cirrhosis of the liver. As detailed in Example 3, chronic treatment of ob/ob mice with CGH significantly reverses steatosis in these subjects. The instant invention thus produces a method for reversing the first “hit” thought to be required for the progression to steatohepatitis and cirrhosis. Further, treatment with the invention of those with steatohepatitis, for whom no efficacious therapy is currently available, would be expected to induce a reversion to a normal (non-steatotic) hepatic state, preventing the progression of pre-cirrhotic hepatitis to cirrhosis.

The invention is further illustrated by the following non-limiting examples.

### EXAMPLES

### Example 1

### Distribution of TSH Receptor Gene Expression

We surveyed RNA samples for TSHR transcript using reverse transcriptase polymerase chain reaction (RT-PCR) amplification. Using standard procedures, RNA samples were isolated from tissues and cell lines, and RT-PCR was run with two separate pairs of primers. The amplified product spans an intron to control for signal arising from genomic DNA contamination. Additionally, TSHR expression was assessed from data in the published literature. Results are described below.

TSH receptor in Adrenal Gland.

RNA from the adrenal cortex carcinoma cell line H295R along with RNA isolated from several adult human normal adrenal glands were found positive for TSHR.

Published literature also documents TSHR transcript in the adrenal gland (Dutton C. M., Joba W., Spitzweg C., Heufelder A. E., Bahn R. S., (1997) Thyroid 6: 879-84).

B. TSH Receptor in a Wide Variety of Cells and Tissue Types.

Extensive panels of RNAs were screened for TSHR and positive expression was found in thyroid, adrenal gland, kidney, brain, skeletal muscle, testis, liver, osteoblast, aortic smooth muscle, ovary, adipocytes, retina, salivary gland, and digestive tract. Similarly, the published literature documents TSHR expression in thyroid, kidney, thymus, adrenal gland, brain, retroocular fibroblasts, neuronal cells and astrocytes (Szkudlinski M. W., Fremont V., Ronin C., Weintraub B. D., (2002) Physiol Rev 82: 473-502 and Dutton C. M., Joba W., Spitzweg C., Heufelder A. E., Bahn R. S., (1997) Thyroid 6: 879-84).

Additionally, as mentioned above, recent reports have documented the presence of TSHR in adipose tissue of humans and rodents. Se, Bell, A., et al. (2000) Am J Physiol Cell Physiol 279, C335-340, and Endo, T., et al. (1995) J Biol Chem 270, 10833-10837

### Example 2

### Expression and Purification of Recombinant CGH

Summary: A Chinese Hamster Ovary (CHO) cell line overexpressing both GPHA2 and GPHB5, the subunits of CGH, was generated and named CHO 180. CHO 180 was found to secrete active, heterodimeric CGH. CGH was purified from the supernatant of CHO 180 using standard biochemical techniques.

A. Generation of CHO 180.

The CGH-producing cell line CHO 180 was generated in two stages. A construct expressing GPHA2, GPHB5 and drug resistance (dihydrofolate reductase) from the CMV promoter was transfected to protein-free CHO DG44 cells (PF CHO) by electroporation. The resulting pool was selected and amplified using methotrexate. Early analysis indicated a high level of GPHA2 expression, but a low level of GPHB5 expression. Therefore, a second construct expressing GPHB5 from the CMV promoter and zeocin resistance from the SV-40 promoter was transfected into the selected, amplified pool by electroporation. After zeocin selection, the final pool (CHO 180) expressed significant levels of both GPHA2 and GPHB5; the proteins were secreted as the non-covalent heterodimer, CGH.

B. Purification of CGH From CHO Culture Supernatant.

CGH was purified from CHO culture supernatant by established chromatographic procedures: first the CGH was captured on a strong cation exchanger, POROS HS50; next it was purified using Hydrophobic Interaction Chromatography with TosoHaas Butyl650S resin; and finally was polished and buffer-exchanged into PBS by Superdex 75 size exclusion chromatography.

C. Cation Exchange Chromatography.

The CHO culture supernatant was 0.2 μm filtered and adjusted to pH 6 and 20 mM 2-Morpholinoethanesulfonic Acid (MES). The CGH in the adjusted supernatant was captured at 55 cm/hr using a 1:2 online dilution with 20 mM MES pH 6 onto a POROS HS 50 column that was previously equilibrated in 20 mM MES pH 6. After loading was complete, the column was washed with 20 column volumes (CV) of equilibration buffer. This was followed by a 3 CV wash with 250 mM NaCl in 20 mM MES pH 6 at 90 cm/hr. Next the CGH was eluted from the column with 3 CV of 500 mM NaCl in 20 mM MES pH 6 at the same flow rate. Finally the column was stripped with steps of 1M and 2M NaCl and then re-equilibrated with 20 mM MES pH 6. The 500 mM NaCl-eluted pool containing the CGH was adjusted at room temperature to 1.0M with (NH₄)₂SO₄ and to pH 6.9 with NaOH for the next step.

D. Butyl 650S Hydrophobic Interaction Chromatography (HIC).

HIC is an adsorptive liquid chromatography technique that separates biomolecules on the basis of net hydrophobicity. The sample is bound to the gel in high salt and then a gradient or step elution of decreasing salt concentration is applied to elute the sample.

The adjusted pool of CGH from the cation exchange chromatography was applied directly at 100 cm/hr to the TosoHaas Butyl650S resin equilibrated in 50 mM NaH₂PO₄ pH 6.9 containing 1.0 M (NH₄)₂SO₄. After loading, the column was washed with 10 CV of equilibration buffer and 10 CV of 50 mM NaH₂PO₄ pH 6.9 containing 0.9M (NH₄)₂SO₄. The CGH was then eluted from the column at 200 cm/hr by reducing the (NH₄)₂SO₄ to 0.5M and collecting 5 CV . This CGH pool was concentrated via ultrafiltration using an Amicon stirred cell with a 5 kDa-cutoff membrane.

E. Size-Exclusion Chromatography.

The concentrated CGH pool was then applied to an appropriately sized bed of Superdex 75 resin (i.e. ≦5% of bed volume) for removal of remaining HMW contaminants and for buffer exchange into PBS. The CGH eluted from the Superdex 75 column at about 0.65 to 0.7 CV and was concentrated for storage at −80° C. using the Amicon stirred cell with a 5 kDa-cutoff ultrafiltration membrane. The heterodimeric protein was pure by Coomassie-stained SDS PAGE, had the correct NH2 termini, the correct amino acid composition, and the correct mass by SEC MALS. The overall process recovery estimated by RP HPLC assay was 50-60%.

Additionally, the CGH polypeptide can be expressed in other host systems. The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from. See, e.g., U.S. Pat. No. 4,956,288. Promoters include those from SV-40 or cytomegalovirus, metallothionein genes (U.S. Pat. Nos. 4,579,821 and 4,601,978) and the adenovirus major late promoter. Within an alternative embodiment, adenovirus vectors can be employed. See, for example, Gamier et al., Cytotechnol. 15:145-55, 1994.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

### Example 3

### Chronic Treatment of ob/ob Mice with CGH

Summary

CGH was administered daily for 28 days to obese male ob/ob mice. Mice were also treated with vehicle saline and thyroxine. Data was obtained for food intake, blood glucose, serum insulin, serum lipids, and serum thyroid hormone levels. At sacrifice, animals were examined for changes in liver pathology, and gross histology. As described below, CGH treatment resulted in decreased post-prandial glucose and insulin levels, and serum triglyceride and cholesterol levels were significantly reduced compared to controls. Thyroid hormone levels were not elevated above the vehicle group, and the control administration of thyroxine did not produce the same results as the CGH treatment group. Evaluation of liver histology sections was performed to examine the effect of CGH-mediated lipolysis on liver steatosis. Prominent liver steatosis typically associated with the ob/ob strain employed in these studies was significantly reversed by CGH treatment, with treated animals exhibiting marked reduction in fat deposition in liver hepatocytes. Thyroid hormone treatment did not produce a significant change in the extent of steatosis.

Treatment Protocol

11-week old male ob/ob mice were individually caged and given a standard lab chow (4% fat) with free access to food and water. Animals were assigned to a treatment group (n=7-8, average weight 54.3±0.3 g /group), kept on a 12 hour dark cycle (6 PM to 6 AM), and injected each day between 7 and 9 AM. Chow consumed by each animal was weighed twice weekly. All animals received treatments IP in an injection volume of 0.1 ml. CGH was administered at 250 μg/kg, dissolved in sterile saline. Thyroxine (T₄) was administered at 1.5 μg/mouse for 4 days, reduced to 1 μg/mouse for 10 days, and returned to 1.5 μg/mouse for the next 14 days. The vehicle controls received sterile saline. CGH was obtained from Genzyme Pharmaceuticals (Thyrogen®, Catalog number 36778; Genzyme Corporation, Cambridge, Mass.), and T₄ obtained from Calbiochem, Inc. (EMD Biosciences, catalog number 61205, San Diego, Calif.) All blood draws were performed by retro-orbital puncture under isoflurane anesthesia.

Food Intake

Food intake did not differ significantly between groups (vehicle 5.9±0.22, CGH 6.3±0.09, and thyroxine 6.1±0.17 grams/day of chow).

Measurement of Serum Thyroxine Levels

After 25 days of treatment as described above, blood was sampled from all treated animals, serum separated, and analyzed for total T₄ by a commercially available kit (Biocheck, Burlingame, Calif.). After 25 days of treatment, the vehicle T₄ levels were 5.14±0.08 μg/dl. The CGH-treated group had T₄ levels of 7.25±1.2 μg/dl, and the thyroxine-treated group had T₄ levels of 9.04±0.47 μg/dl. The thyroxine treatment group had levels significantly higher than vehicle controls (p<0.001).

Treatment Effects on Glucose and Insulin Levels

Subject animals were fasted for 4 hours at the beginning of the light cycle, and serum was obtained at treatment day 25 under isoflurane anesthesia. Serum Glucose levels were determined with the Cholestech LDX blood analyzer (Cholestech Corporation, Hayward Calif.), and serum insulin levels by ELISA. Serum glucose levels for vehicle and thyroxine treated groups were 306±22 and 295±23 mg/dl, respectively. Serum glucose levels for the CGH treated animals were significantly lower, 251±10 mg/dl, (p<0.05). Serum insulin levels for the vehicle and thyroxine treated groups were 33.5±1.8 and 25.9±1.7 ng/ml, respectively. Serum insulin levels in the CGH treatment group were significantly decreased to 14.2±3.6 ng/ml, (p<0.001).

Serum Lipid Analysis

Subject animals were fasted for 4 hours at the beginning of the light cycle, and serum was obtained at treatment day 25 under isoflurane anesthesia. Triglyceride and total cholesterol levels were determined with the Cholestech LDX blood analyzer. Serum triglyceride levels for the vehicle controls were 143.7±23 mg/dl. The serum triglycerides in the CGH-treated group were lower at 100±14.7 mg/dl, (p=0.09), and the triglycerides in the thyroxine treated group were higher than the vehicle controls at 198±43 mg/dl, (p=0.26). Total cholesterol levels in the vehicle-treated and thyroxine-treated groups were 198±13 and 194±15 mg/dl, respectively. Total cholesterol average of the CGH treatment group was significantly lower at 104±8.8 mg/dl, (p<0.01).

Liver Steatosis

Liver sections were dissected from all treatment groups described above and mounted in paraffin following fixation with NBS-formalin. Sections were mounted and stained with hematotoxylin and eosin (H&E) for visualization of hepatic structural changes. The extent of liver steatosis was evaluated on a four-point scale, from 0 to 3, with zero displaying no signs of liver steatosis, and a score of 4, represented pronounced macrovesicular and microvesicular steatosis. The averages of the groups (n=4) showed significant differences in the extent of steatosis as judged by the size of the lipid inclusions and the integrity of the hepatocyte structure visible in the sections. Average scores given to the groups were vehicle (4), thyroxine (3), and CGH (1.5). The CGH treated animals exhibit a loss of vacuolarization represented by the accumulation of lipid droplets in the hepatocytes. The CGH treated sections appear to have regained hallmarks of normal hepatocyte architecture in only 28 days of treatment, with areas containing hepatocytes without lipid-laden inclusions in the cytoplasm of the cell.

1. A method for treating steatosis in a mammal, comprising administering to the mammal a pharmaceutically effective amount of a CGH polypeptide, wherein administration of the polypeptide results in a reduction in the steatosis.

2. The method of claim 1, wherein said mammal is obese.

3. The method of claim 1, wherein said mammal is type-2 diabetic.

4. The method of claim 1, wherein said mammal has metabolic syndrome.

5. The method of claim 1, wherein said mammal is hyperlipidemic.

6. The method of claim 1, wherein said mammal is insulin resistant.

7. A method for treating steatohepatitis in a mammal, comprising administering to the mammal a pharmaceutically effective amount of a CGH polypeptide, wherein administration of the polypeptide results in an improved steatohepatitic state.

8. The method of claim 7, wherein said mammal is obese.

9. The method of claim 7, wherein said mammal is type-2 diabetic.

10. The method of claim 7, wherein said mammal has metabolic syndrome.

11. The method of claim 7, wherein said mammal is hyperlipidemic.

12. The method of claim 7, wherein said mammal is insulin resistant.

13. A method for preventing steatohepatitis in a mammal, comprising administering to the mammal a pharmaceutically effective amount of a CGH polypeptide, wherein administration of the polypeptide maintains or reduces the steatohepatitis.

14. The method of claim 13, wherein said mammal is obese.

15. The method of claim 13, wherein said mammal is type-2 diabetic.

16. The method of claim 13, wherein said mammal has metabolic syndrome.

17. The method of claim 13, wherein said mammal is hyperlipidemic.

18. The method of claim 13, wherein said mammal is insulin resistant.

19. A method for preventing cirrhosis of the liver in a mammal, comprising administering to the mammal a pharmaceutically effective amount of a CGH polypeptide, wherein administration of the polypeptide maintains or reduces the cirrhosis.

20. The method of claim 19, wherein said mammal is obese.

21. The method of claim 19, wherein said mammal is type-2 diabetic.

22. The method of claim 19, wherein said mammal has metabolic syndrome.

23. The method of claim 19, wherein said mammal is hyperlipidemic.

24. The method of claim 19, wherein said mammal is insulin resistant.

25. A method for treating cirrhosis of the liver in a mammal, comprising administering to the mammal a pharmaceutically effective amount of a CGH polypeptide, wherein administration of the polypeptide reduces the cirrhosis.

26. The method of claim 25, wherein said mammal is obese.

27. The method of claim 25, wherein said mammal is type-2 diabetic.

28. The method of claim 25, wherein said mammal has metabolic syndrome.

29. The method of claim 25, wherein said mammal is hyperlipidemic.

30. The method of claim 25, wherein said mammal is insulin resistant.
